(19) United States
(12) Patent Application Publication
(10) Pub. No.: US 2010/0087834 A1
(43) Pub. Date: Apr. 8, 2010

(54) BIPOLAR INSTRUMENT AND METHOD FOR ENDOSCOPIC CONTROLLED SHORTENING AND/OR FRAGMENTATION OF STENTS ARRANGED IN GASTROINTESTINAL TRACT IN THE TRACHEOBRONCHIAL SYSTEM OR IN OTHER HOLLOW ORGANS

(76) Inventors: Florian Eisele, Tubingen (DE); Daniel Schäller, Tubingen (DE); Matthias Voigtländer, Gomaringen (DE)

Correspondence Address: DICKSTEIN SHAPIRO LLP, 1825 EYE STREET NW, Washington, DC, 20006-5403, US

(21) Appl. No.: 12/524,440

(22) PCT Filed: Jan. 25, 2008
(86) PCT No.: PCT/EP08/00602

§ 371 Date: Dec. 17, 2009

(30) Foreign Application Priority Data
Jan. 25, 2007 (DE) 10 2007 003 838.2

(51) Int. Cl. A61B 17/00

(52) U.S. Cl. 606/108

(57) ABSTRACT

A bipolar instrument and a method for endoscopically controlled shortening and/or fragmentation of a stent located in the gastrointestinal tract, in the tracheobronchial system or in other hollow organs. The instrument includes a first electrode and a second electrode arranged at a distal end of the instrument and connected to and receiving current provided by a power source, and protective means connected to the electrode means. At least one wire of the stent can be severed at a particular location by the instrument. The protective means separates the wire from tissue of the gastrointestinal tract, tracheobronchial system or other hollow organs and/or secures the wire to the instrument during the severing of the wire. The instrument and the method minimize damage to tissue and risk to the patient during machining of stents.

### FIELD OF THE INVENTION

The disclosed embodiments relate to a bipolar instrument and to a method for endoscopically controlled shortening and/or fragmentation of stents located in the gastrointestinal tract, in the tracheobronchial system or in other hollow organs.

### BACKGROUND

Stents are increasingly used for the palliative treatment of stenosing tumors or scar tissues, for covering or closing anastomotic insufficiencies, fistulas and the like, for bridging necrotic cavities, etc. of the gastrointestinal tract or the tracheobronchial system.

FIG. 15 illustrates an example of a stent 70. Stents are resilient tubes which are plaited, knitted or otherwise made of special wires 71, for example metal wires, and have more or less large meshes. The purpose of stents of this type is to widen, as a result of their radially acting resilient force, the lumen of pathologically narrowed hollow organs, for example the oesophagus as a consequence of stenosing tumor growth.

When implanted correctly, stents abut tightly against the respective organ wall or against the pathological tissue of the organ wall with sufficient radial spring force to ensure the passage of solid, liquid and/or gaseous substances through the hollow organ in question. However, stents fulfil their purpose only when and for as long as they keep free the lumen required for the functioning of the particular hollow organ.

However, if a stent is implanted incorrectly, damaged during or after implantation or is otherwise inadequate, to may not be able to fulfil its purpose. In this case, it may be necessary to shorten this stent and/or to completely explant it or to remove it from the hollow organ in question. This removal can be very difficult because an advantage of stents, namely the effective and secure force-fitting fixing to the organ wall, prevents explantation of the stent as well. The explantation of stents is particularly problematic when the stents lie in curves of hollow organs and/or are deformed or even tumor tissue or other tissue has grown inward from the outside through the mesh of the stent. If the stent abuts the organ wall too tightly or if tissue has grown into the meshes thereof and/or if the stent is deformed, so that it cannot be explanted as a whole in one piece, then it has to be divided into sufficiently small explantable fragments.

In the past, thermal methods have been used for the shortening of stents. In the thermal methods, the metal wires of a stent are heated to the melting temperature thereof at the points suitable for shortening or fragmentation and in this way are severed. Endoscopically applicable LASERs, in particular Nd:YAG-LASERs or argon plasma, are used for this purpose. Nevertheless, the Nd:YAG-LASERs and argon plasma applicators previously available for endoscopic use are intended for thermal haemostasis and/or for thermal devitalisation, coagulation, desiccation, but are not designed for fusing metal wires. Both of these methods can cause accidental heat damage to tissue directly adjacent to the point of application and/or also to tissue more remote therefrom. The application of Nd:Yag-LASERs is also expensive and subject to adherence to extensive safety regulations. In addition, only metal wires can be machined using these methods.

It is the object of the disclosed embodiments to provide endoscopically applicable instruments used for and a method for shortening and/or fragmentation of stents located in the gastrointestinal tract, in the tracheobronchial system or in other hollow organs. The instruments and the method are intended to avoid damage to tissue directly adjacent to the point of application and also to tissue as far as possible therefrom. It should also be possible to carry out the machining and removal of the stents as simply as possible for the operator and without risk to the patient.

### SUMMARY

Disclosed embodiments include a bipolar instrument for endoscopically controlled shortening and/or fragmentation of stents located in the gastrointestinal tract, tracheobronchial system or in other hollow organs, which instrument comprises the following:
an electrode means which is arranged at a distal end of the instrument and has at least a first electrode and a second electrode for passing a current from a power source through at least one wire of the stent and/or for forming electric arcs between the first electrode and the at least one wire and/or between the first electrode and the second electrode, so that the wire can be severed by heating and fusing,
a protective means which is embodied and mechanically connected to the electrode means such that the wire can thus be separated and/or set apart from tissue of the gastrointestinal tract, tracheobronchial system or other hollow organs and/or secured to the instrument during the passing of the current and/or during the formation of electric arcs.

In the disclosed embodiments, a single instrument and a correspondingly suitable method are used to detach individual wires (or small groups of wires) of the stent from the tissue adjoining them, so that the introduction of the high-frequency current and/or the forming of arcs for heating the wire or stent fragments or else a plurality of wires can be carried out in a precise manner and damage to the tissue originally abutting the wire is minimized. In addition, due to direct contact of electrodes and wire or due to the formation of arcs between the electrode and wire and the formation of arcs between the electrodes, both metallic wires and non-metallic wires (e.g., plastic wires) can be heated and melted. The bipolar arrangement therefore allows the machining of stents made of different materials, not just metal.

An electrode means that has at least two electrodes is arranged at the distal end of the instruments. For the direct heating of stent wires, the electrode touches the stent wires. For the indirect heating of stent wires, the electrode is set apart from the stent wires to generate the electric arcs required for the indirect heating of stent wires. Arcs can be directed directly onto the wire or can be generated between the electrodes, so that the heat of the arcs heats a stent fragment or plurality of wires located in the immediate vicinity thereof. The arcs being generated between the electrodes is particularly important for non-metallic stents in order to be able to fragment or shorten these too. For the electric heating of a metallic stent wire, it is possible to pass an electric current through the wire which heats the wire directly, i.e. from the inside, or to direct an electric arc onto the wire which heats the stent wire additionally or predominantly indirectly, i.e. from the outside. Arcs generated between the electrodes are also suitable for fusing metallic wires. It should be noted at this point that the term “wire” is intended to focus not only on metallic wires; on the contrary, the term refers to any type of stent materials (including for example stents made of plastic).

For safety reasons, the source used for the electrical energy is preferably a generator of an electrosurgical apparatus which generates high-frequency electric alternating current. The generator is preferably short circuit-proof, so that machining of the low-resistance stent wires can be carried out without difficulty.

Since the instrument according to the disclosed embodiments is provided for the machining of stents (and not, for example, for the treatment of tissue of a patient), the power source used may also be a DC power source or low-frequency AC power source. It might be necessary to ensure in this case that no current greater than 10 μA flows to the earth potential; the generator would therefore have to be insulated accordingly.

Preferably, at least the power source is embodied such that it can be allocated to a control means for controlling the current and/or arc required for heating and fusing the wire, the control means being embodied such that the current can be controlled or regulated for automatically controlled severing of the wire. Preferably, an arc monitor and/or a current monitor can be allocated to the control means, so that the current can be controlled or regulated as a function of a detected arc or as a function of a detected current value. Thus, for example owing to the detection of arcs, the corresponding further course of the machining can be controlled or regulated, so that an operator does not have to make decisions in this regard.

In the case of a DC power source with which it is necessary to monitor the current intensity, detection of currents which occur can facilitate the operability of instruments according to the disclosed embodiments. Thus, the control means can be embodied such that the currents are measured and the supply of current is interrupted on occurrence of a threshold value or limit value. Damage to the patient may thus be avoided.

In one example embodiment, at least the electrode means and the protective means are embodied as an effector. The effector is arranged at a distal end of the instrument. A gripping means, which improves handling of the respective instrument, can, if required, be arranged at a proximal end of the instruments according to the disclosed embodiments. The instrument can, if appropriate, be embodied such that the effector can be handled and moved independently in relation to the instrument. This makes the instrument easier to handle.

Advantageously, the instrument may include a rigid or flexible shaft or catheter, the shaft or the catheter being embodied such that it can be brought up to the stent through an instrument channel of a rigid or flexible endoscope. Minimally invasive interventions generally subject the patient to only slight stress.

In one example embodiment, the shaft or the catheter is embodied as a pipe or as a tube with a respective lumen as a supply arrangement for supplying a fluid, in particular a gas and/or a liquid, to the electrodes and/or the protective means and/or the hollow organ. Preferably, the supply arrangement is arranged relative to the electrodes, in particular surrounding the electrodes, such that the electrodes and/or the electrode means and/or the protective means can be cooled by the fluid supplied and/or the passing of the current through the wire and/or the forming of arcs is carried out under a protective gas atmosphere. The effector can be part of the supply arrangement and thus of the pipe or tube, wherein both elements can be made (in a substantially insulating manner) of different materials.

In one example embodiment, the effector includes the supply arrangement, i.e. the lumen. In other words, the shaft could be embodied such that gas is supplied only into the effector. Conventionally, however, the fluid is supplied from a proximal end of the instrument. By supplying a cooling fluid, it is possible, for example, to prevent the electrodes and/or the entire distal end of the instrument, including the entire effector, from becoming too hot, as a result of electric arcs. At least the distal end can effectively be cooled by a suitable fluid during operation of the instrument, since the supply arrangement is arranged in a corresponding manner relative to the electrode (e.g., surrounding the electrode). For this reason, the electrodes are configured within the shaft or the catheter such that the coolant can rinse around them. For example, the first electrode may include a partial helix in order to be secured in a form-fitting manner in the shaft or catheter. The coolant used can be a gas (e.g., air or a noble gas such as argon), which may be supplied through the shaft or catheter from the proximal end of the instrument.

When using the instruments according to the disclosed embodiments in proximity to combustible substances (e.g., plastic-coated stents), it may be expedient to introduce an inert gas, in particular argon, via the supply arrangement, especially in the region of the electric arcs. This can be carried out in the same manner as the introduction of coolants. In this way, undesirable gases located in hollow organs can also be kept away from the region of action of the arc. In individual cases, it may therefore be advantageous to generate electric arcs not in air but rather in a protective gas atmosphere (protective gas, noble gas), especially if combustible material is present in the region of the arc formation, so that the wire is heated in a protective gas atmosphere.

The flexible shaft or catheter may also be embodied as a rod element made of solid material. In this embodiment, the electrode means is arranged, and in particular embedded, at the distal end of the shaft or catheter. The electrodes are embedded into the shaft or catheter material such that they are accessible at active regions for machining the stent wires. Also, according to this example embodiment the shaft or catheter may comprise the effector. The rod element and effector may be made of different materials.

The instruments according to the disclosed embodiments are then for example embodied without an explicit lumen, if no fluid has or is to be supplied to the effector.

Preferably, the shaft or catheter is made of ceramic, plastic or a similar insulating material. Thus, the electrodes can be embedded set apart and insulated from one another. It is also possible to make only the effector of ceramic, while the remaining shaft or catheter is made of plastic.

A protective means is provided at the distal end of either the instrument or the effector. The protective means serves to separate or to set apart the stent or a wire of the stent from patient tissue on which it is resting or by which it is surrounded, and, if appropriate, to secure it to the instrument. The protective means is electrically insulating and made of heat-resistant and arc-resistant material. It is thus possible to separate a selected stent wire reliably and in a simple manner from the tissue in order to prevent the stent wire from being cooled by water-bearing tissue and to ensure that the wire is reliably received in the instrument for machining thereof.

The protective means is preferably embodied and arranged relative to the electrodes such that the wire can be held at a predetermined spacing from the first electrode and/or from the second electrode. For the indirect heating of stent wires by electric arcs, at least the protective means therefore has a spacer which is configured such that the first electrode (in principle the active electrode), when used as intended, does not directly contact stent wires, but is at a minimum spacing therefrom. Electric arcs are produced between the electrode and the stent wire, at a sufficiently high electrical voltage to heat the stent wires to the melting temperature thereof.

Preferably, the protective means has a means for threading the wire into the protective means and/or for separating and/or setting apart the wire from the tissue. This means may be spatula-shaped, finger-shaped, spoon-shaped or other similar shape. This means allows the wire to be pushed or pulled between stent wires abutting tissue and tissue to a sufficient distance so that the wire is received in the protective means and thus raised from the tissue and positioned for heating. These spatula-shaped or finger-shaped or similarly shaped means can be adapted in their shape and size to be compatible with the various existing stents as well as future models of stents. Means of this type are handled in particular in the axial direction of the instrument. Thus, the instrument as a whole can be displaced in the axial direction or else the instrument is constructed such that only the protective means and/or the means can be handled. If appropriate, the effector can also be embodied so as to be movable per se.

A further embodiment of a means for threading in and/or separating and/or setting apart stent wires is screw-shaped, helical or corkscrew-shaped in its configuration. In this way, stent wires can be raised from the tissue such that the means rotates between the stent wire and tissue, i.e. is screwed. In other words, it can be screwed and/or slid in a substantially turning or rotating movement under the at least one wire.

This means can be optimally adapted in shape, size and handling in accordance with the wire guidance of the particular stent. What is important in this case is primarily the fact that this means is suitable to set apart the stent wires to be severed during the direct or indirect heating of water-bearing tissue.

Preferably, the means is embodied such that it allows simultaneously a plurality of wires to be threaded in and/or separated and/or set apart from the tissue. It is thus possible for even relatively large stent fragments to be separated off and melted off from the stent.

One embodiment provides for the protective means to have at least one guide which is embodied such that the wire slips into the guide and can be fixed therein during the pressing-on of the instrument and/or the sliding or turning of the means and/or of the instrument. It is thus possible to simply and securely position the wire relative to the electrodes. If the guide is embodied as at least one notch, then the wire can be received in this notch in a simple manner. The guide, in particular the notch, has advantageously a region in which the wire can be positioned in an end position for safe machining by means of the active electrode.

The protective means may have a different pitch for forming the notch, which allows a different clamping function to be produced. Depending on the formation of the notch and purpose of application, various holding effects of the wires may therefore be attained. For example, a smaller notch angle would ensure a greater holding force of the positioned wire, while a larger angle allows easier detachability after the separating process.

Preferably, the guide is embodied such that the received wire can be held at the predetermined spacing (at least) from the first electrode. That is to say, the wire or else the stent fragment (or a plurality of wires) may be received in the guide only far enough in so that a suitable spacing can be adhered to for forming arcs between, for example, the first electrode and the wire.

The protective means or the guide can however also be embodied such that the wire received in the protective means is correctly positioned for directly introducing current. In any case, the protective means and/or the guide is embodied such that the wire or the stent fragment can be brought in a position suitable for machining and be held therein.

The protective means or the guide can therefore be embodied such that the wire can be arranged between the electrodes or at least in direct proximity and can thus be severed via direct or indirect heating.

Advantageously, the protective means is embodied such that, when the wire is received therein, the spacing between the first electrode and the wire is smaller than a spacing between the first electrode and the second electrode. A spacing between the wire and electrode is required for forming arcs. Depending on the embodiment of the instrument, the spacing between the wire and electrode or between the electrodes are to be designed such that the arcs are produced between the desired positions (e.g., between the first electrode and wire). Only the appropriate design of the spacing ensures efficient machining of the stents. In addition, the maximum voltage is to be kept sufficiently low that ignition never occurs by way of the spacing between the electrodes.

It may also be desirable to generate the arcs between the electrodes in order to sever a wire positioned nearby, in particular owing to the heat of the arcs. In this case, it would have to be possible to position a metallic wire set apart accordingly from the electrodes in order to avoid an undesirable formation of arcs between the wire and electrodes.

The effector preferably includes a sleeve or a holder for holding the electrodes that is made of electrically non-conductive material (insulating material) such as, for example, ceramic. In one disclosed embodiment, the protective means is connected securely to (or formed as one piece with) the sleeve or the holder. The lumen is embodied at least in the sleeve. The electrodes are then fitted or embedded into the sleeve, as will be described hereinafter in greater detail. The sleeve-shaped embodiment of the effector allows the formation of a lumen, while the electrodes are preferably embedded into an effector (holder) made of solid material and form regions which are active at defined regions.

In one embodiment the electrodes are embodied on the pipe or the tube such that the first electrode is arranged in the lumen and the second electrode is arranged coaxially with the first electrode, set apart therefrom. In other words, the two electrodes extend coaxially with one another in the axial direction of the instrument. The first electrode can then be embodied with the above-described fastening helix and arranged substantially centrally in the lumen. The second electrode then surrounds the first electrode, for example, in a tubular manner, wherein gas can be supplied between the two electrodes. The tubular electrode can in this case itself be part of the shaft or the catheter or the effector or is embedded into the insulating material. In the latter case, both electrodes are ultimately held by the effector. A wire received in the protective means, in particular in the guide, can then be machined by means of the electrodes such that, for example, arcs from the first electrode are directed onto the wire. In this case, the guide ensures suitable spacing of the wire and first electrode. The path of the current extends from the power source via the first electrode and the arc to the wire and up to the second electrode, since the wire rests on the second electrode via the guide. The protective means is embodied such that the wire rests on the second electrode via the protective means. The wire then returns to the power source via the second electrode. The protective means or the guide can also be embodied such that direct contact of (both) the electrodes and wire causes severing of the stent fragment.

In yet another embodiment the electrodes are arranged on the pipe or tube or on the rod element such that the first electrode and the second electrode are embedded, set apart from one another, in the pipe or tube or in the rod element such that they each form an active region at a distal end of the instrument. For example, only the end regions of the electrodes are available for the action of current in this case.

The arrangement of the electrodes and the protective means relative to one another allows direct contact of the wire and electrodes, so that the received wire or the wires or the stent fragment abuts or abut against the electrodes. In this case, the electrodes can be arranged such that the wire is touched by the electrodes in each case at the same cross section or at various cross sections. In so far as the electrodes are embedded into the effector, they must be accessible at least one point for the introduction of current and/or formation of arcs. This is possible via the active regions.

Preferably, the electrodes are arranged on the pipe or tube such that the first electrode and the second electrode are embedded, set apart from one another, in the pipe or tube such that their active regions at least partly surround the lumen. In this example embodiment the effector or at least the distal end of the instrument (generally the entire shaft or catheter) is embodied as a tubular element with the electrodes arranged within an insulation layer of the effector or the instrument and, for example, substantially opposing one another. A fluid may be supplied to the machining point, i.e. for example to the electrodes, via the lumen located between the electrodes. As described hereinbefore, rinsing liquids or similar fluids can also be supplied.

Preferably, the first electrode and/or the second electrode each comprise at least one raised region extending in the direction toward the respectively opposing electrode in order to form the arcs. With opposing tips, arcs can be formed very much more easily, as less voltage is required to be provided. In particular, the current can in this case be controlled or regulated such that arcs are formed only via the regions provided for this purpose between the electrodes (in this case for example the tips), while the other electrode regions are not active.

Advantageously, a wire can be positioned in proximity to the tips via the protective means or via the guide such that it is melted predominantly by the heat of the arc. A special mount can be provided, if appropriate, which ensures, in addition to guidance, suitable positioning. Non-metallic wires may thus also be machined.

The electrodes are in one embodiment made of a high temperature-resistant material, for example tungsten, and/or are designed in such a way, for example so as to be more solid than the stent wires to be severed, that they do not melt when used as intended. Lanthanated tungsten wire is particularly suitable in this case.

On application of direct current, it might be necessary to ensure that one-sided electrode wear can take place here. Thus, for example, the active electrode would lose material, while deposits build up at the opposite electrode. This could, if appropriate, be counteracted by an unsymmetrical configuration of the electrode means (thicker first electrode, thinner second electrode).

In one embodiment the protective means and/or the means for threading in and/or separating and/or setting apart the wire from the tissue comprise at least one holding means, in particular a hook element, for receiving and securing the wire, the stent fragment or the stent on the instrument. That is to say, a means is provided, which prevents for example a wire, once received or threaded in, from slipping out of the protective means or the means for threading in and/or separating and/or setting apart the wire from the tissue. For this purpose, the protective means can have as the holding means at least one hook element, i.e. for example a barb, which ensures secure holding of the wire in the protective means. The barb therefore allows wires to be “trapped” and drawn away from the tissue.

Preferably, the holding means has a large number of barbs which (even in the event of non-precisely defined handling of the instrument or the means) are arranged, for securely receiving the wire, the stent fragment or the stent, on the protective means, set substantially uniformly apart from one another. If the effector has for example a circular cross section, the barbs are arranged preferably radially symmetrically.

According to the disclosed embodiments, the holding means can be arranged on the means for threading in and/or separating and/or setting apart the wire from the tissue. The holding means supports the protective means or the means for threading in and/or separating and/or setting apart the wire from the tissue.

It may be advantageous to embody the holding means so that it can move for movement of the wire, the stent fragment or the stent itself. The barb would then be movable, for example relative to the protective means, and could for example be brought up in the direction of the guide. This would also facilitate positioning of the wire, the stent fragment or even the stent.

If the wire, stent fragment or stent can be secured via the holding means, then they can be removed in a controlled manner from the hollow organ and thus from the operating area by means of the holding means, i.e. extracted from the hollow organ.

The instruments according to the disclosed embodiments therefore allow a stent fragment or the stent to be removed from the gastrointestinal tract, tracheobronchial system or other hollow organs. This refers to complete removal from the patient's body. If the instruments according to the disclosed embodiments allow the stent fragment, which has been separated off from the stent, to be removed at the same time from the area of use, once a fragment has been separated off, it does not have to remain in the hollow organ until it can be removed from the area by means of a further instrument, for example a pair of pliers. In this respect, the instrument is embodied such that it can be used to carry out also complete removal of the fragment or of the stent as a whole.

In terms of the method, the object is achieved in that, in a method for endoscopically controlled shortening and/or fragmentation of stents located in the gastrointestinal tract, tracheobronchial system or in other hollow organs with a bipolar instrument having at least a first electrode and a second electrode and a protective means which is mechanically connected to the electrodes, the following steps are provided:
a) bringing the instrument into the hollow organ and to the stent;
b) separating and/or setting apart at least one wire from tissue of the gastrointestinal tract, tracheobronchial systems or other hollow organs by inserting or screwing in the protective means between the wire and tissue and/or securing the wire to the instrument by means of the protective means and positioning the at least one wire at least in proximity to the electrode means by means of the protective means such that a current can be passed through the wire of the stent and/or electric arcs can be formed between the first electrode and the wire and/or between the first electrode and the second electrode;
c) passing the current from a power source by means of the electrode means into the at least one wire and/or forming electric arcs between the first electrode and the wire and/or between the first electrode and the second electrode and thereby severing the wire; and
d) repeating steps b) and c) for shortening and/or fragmenting the stent.

On use of the instruments according to the disclosed embodiments, this method allows at least one stent wire to be melted off and thus detached from the stent. In order now to melt a plurality of wires of the stent, which is positioned in the hollow organ, from the stent and thus to shorten or to trim or to fragment the stent or even to explant the stent as a whole, steps b) and c) are to be repeated accordingly often.

### BRIEF DESCRIPTION OF THE DRAWINGS

The disclosed embodiments will be described hereinafter based on example embodiments which will be explained in greater detail with reference to the enclosed drawings.

FIG. 1 illustrates a cross-sectional view of a distal end of a disclosed embodiment of the instrument (along the line I-I from FIG. 2).

FIG. 2 is a side view of the distal end of the instrument according to the embodiment of FIG. 1.

FIG. 3 illustrates a side view of the distal end of the instrument according to another disclosed embodiment.

FIG. 4 illustrates a side view of the distal end of the instrument according to another disclosed embodiment.

FIG. 5 illustrates a cross-sectional view of the instrument according to another disclosed embodiment.

FIG. 6 illustrates a view of the distal end of the instrument along the line VI-VI of FIG. 5.

FIG. 7 illustrates a cross-sectional view of the distal end of the instrument according to another disclosed embodiment.

FIG. 8 illustrates a view of the distal end of the instrument along the line VIII-VIII of FIG. 7.

FIG. 9 illustrates a view of the distal end of the instrument along the line IX-IX of FIG. 8.

FIG. 10 illustrates a cross-sectional view of the distal end of the instrument according to another disclosed embodiment.

FIG. 11 illustrates a side view of the distal end of the instrument according to another disclosed embodiment.

FIG. 12 illustrates a side view of the distal end of the instrument according to another disclosed embodiment.

FIG. 13 illustrates a side view of the distal end of the instrument according to another disclosed embodiment.

FIG. 14 illustrates a detail view of the instrument according to the disclosed embodiments (e.g., according to FIG. 1) with a gripping means.

FIG. 15 illustrates an example of a stent.

### DETAILED DESCRIPTION

In the subsequent description the same reference numerals will be used for like and equivalent parts.

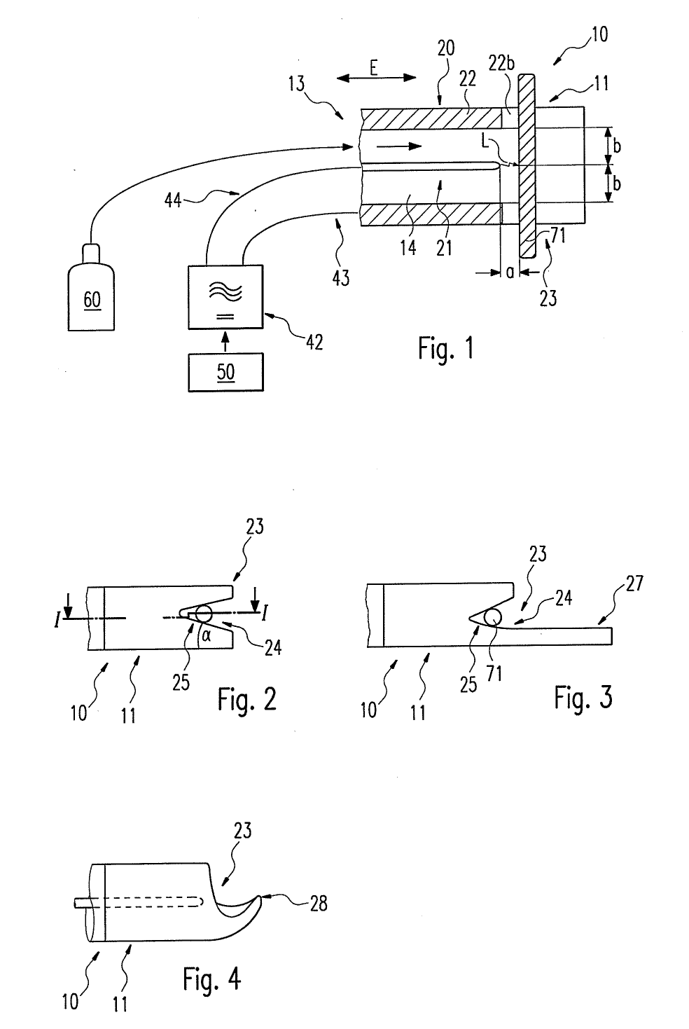

FIG. 1 illustrates one disclosed embodiment of the instrument 10. In FIG. 1, a cross-sectional view along the line I-I from FIG. 2 of the distal end 11 of the instrument 10 is illustrated. FIG. 2 is a side view of the distal end 11 of the instrument 10. Instruments according to the disclosed embodiments may be used to shorten and/or fragment stents located in the gastrointestinal tract, tracheobronchial system or in other hollow organs.

As previously discussed, explanting or removing a stent from a hollow organ can be very difficult if the stent abuts the organ wall too tightly or if tissue has grown into the meshes thereof and/or if the stent is deformed. In these cases, the stent cannot be explanted as a whole in one piece and instead must be divided into sufficiently small explantable fragments. The instruments 10 according to the disclosed embodiments can be used for this purpose. These instruments 10 are used to heat the stent wires so that they melt at a substantially planned location (separation region).

It should be noted that the instrument 10 is embodied both for receiving a single wire 71 and for receiving stent fragments or a plurality of wires. Although reference is made hereinafter only to “wire,” the term “stent fragment” is nevertheless also included or there may also be a plurality of wires. Moreover, the instruments according to the disclosed embodiments also allow stents to be grasped as a whole and, if appropriate, also to be removed as a whole from the corresponding hollow organ. The instrument would then serve as a type of pair of pliers. Wires or wire fragments may also be machined and removed by means of the instrument.

The term “wire” is also not restricted to metallic wires. The instruments according to the disclosed embodiments may also be used for machining plastic wires or wires formed of other materials, for example coated wires.

The wires are heated either directly, by introducing current into the wires, or indirectly, by utilising the heat from arcs L. An electrode means is provided for heating the wires. The electrode means is embodied as a bipolar arrangement and consists of at least a first and a second electrode 21, 22.

In the embodiment illustrated in FIGS. 1 and 2, the instrument 10 is embodied as either a rigid or flexible shaft or catheter 13, so that the shaft or the catheter can be brought up to the stent 70. This may be accomplished, for example, through an instrument channel of a rigid or flexible endoscope (not shown). The shaft or catheter 13 is tubular and therefore includes a lumen 14.

The first, rod-shaped electrode 21 is arranged (substantially centrally) in the lumen 14 of the shaft or catheter 13 in the direction of extension E of the instrument, while the second electrode 22, as a tubular element, is arranged coaxially with the first electrode 21, set apart therefrom. The electrodes 21, 22 thus extend in an axial direction E of the instrument and are connected to a power source 42 via current supply means 43, 44. The power source 42 provided is preferably a high-frequency AC power source, i.e. a high-frequency generator. It is also possible to use a DC power source or a source for low-frequency current since, in this case, the supply of current is not provided into the human (or else animal) body. The power source 42 shown in FIG. 1 illustrates that both alternating current and direct current can be used.

Furthermore, the instrument 10 is connected to a gas source 60, so that a gas can be brought up to the electrodes 21, 22 via the lumen 14 (the arrow drawn in the lumen indicates the direction for the supply of fluid). In individual cases, it may be advantageous to carry out the introduction of current into the wire 71 under, for example, a protective gas atmosphere in order to keep inflammable gases in the hollow organs away from the region of action of the electrodes. This is especially advantageous when arcs L are to be used. Cooling fluids, rinsing liquids or other fluids may also be supplied via the lumen 14. This allows, for example, the electrode region or the distal end 11 of the instrument 10 to be cooled by means of the cooling fluid, thereby avoiding overheating of the distal end of the instrument and resulting damage to tissue surrounding the stent 70.

According to FIG. 1, a control means 50 is provided for controlling the current and/or arc by activating, for example, the power source. The control means includes a current monitor and/or an arc monitor. The controller allows the current to be controlled or regulated such that the operator does not have to make decisions in this regard, and instead the course of the machining is carried out in an optimized manner. After detection of an arc, it is possible to set a defined time period over which the wire is to be exposed to the current and/or heat. It is also possible to control the voltage in order to allow adequate introduction of current. If a DC power source is used, it is advantageous to monitor the current. Thus, the control means 50 can measure the currents and, on the occurrence of a threshold value or limit value, the supply of current to the electrodes 21, 22 is interrupted.

The power source (i.e. the high-frequency generator) and control means can be jointly accommodated in a (high-frequency) surgical apparatus.

The instruments 10 according to the disclosed embodiments have a protective means 23 by means of which the wire 71 can be separated and/or set apart, during the passing of the current and/or during the formation of arcs L, from tissue of the gastrointestinal tract, tracheobronchial systems or other hollow organs. As seen in FIG. 2, the protective means 23 may be a notch-shaped recess, so that the wire 71 can be raised from the tissue and received in the notch and positioned therein for machining with the instrument 10. The notch forms a guide 24 of the protective means 23. The notch can be formed at different angles, so that either the force for holding the received wire 71 (or stent fragment) can be increased or removability from the notch is facilitated. The angle α is therefore variable—an increased clamping function is produced at a low α and easier detachability of the machined wire 71 is provided at a greater α.

The electrode means 21, 22 and the protective means 23 form an effector 20 at the distal end 11 of the instrument 10. The effector 20 is embodied in the embodiment of FIG. 1 in a sleeve-shaped manner. The effector 20 is made of electrically, and preferably also thermally, insulating material. In addition to the sleeve form shown in FIG. 1, the effector 20 may also be embodied as solid material. The effector 20 is, generally, a holder in which the electrodes 21, 22 are arranged. Usually, the electrodes 21, 22 are embedded in the effector 20. In the case of a sleeve-shaped embodiment (tubular), the effector 20 may be a ceramic tube in which the electrodes 21, 22 are embedded or clamped. Owing to the sleeve shape, the effector 20 (as the distal end of the instrument) also forms the lumen 14.

The effector 20, and thus the distal end of the shaft or catheter, carries both the electrodes 21, 22 and the protective means 23 and is, in the embodiment of FIG. 1, connected in one piece with the protective means 23. The protective means 23 is embodied so as to be electrically and thermally insulating in order to prevent damage to tissue abutting the stent 70 by the introduction of current and/or heat. In this embodiment, the second electrode forms the sleeve shape of the effector 20, wherein the sleeve can be insulated toward the outside. At a proximal end 12 of the instruments 10 according to the disclosed embodiments there can be arranged, if required, a gripping means (not shown here) which improves handling of the particular instrument.

As shown in FIG. 1, the protective means 23 has the guide 24 or in this case the notch, so that the wire 71 or stent fragment can be guided into the protective means 23. By pressing the instrument 10 onto the implanted stent 70 and/or the surrounding tissue, the wire 71 can be received in the guide 24, i.e. in this case in the notch, and may thus be brought into a suitable position for machining. In this example embodiment, the first electrode 21 is arranged in the effector 20 (i.e., in the lumen 14 of the instrument 10) and the protective means 23 or the guide 24 is embodied such that a received wire 71 can be positioned set apart from the first electrode 21. At the same time, the wire 71 rests on the second electrode 22 via the notch. Current and voltage can now be controlled and regulated such that arcs L can be formed between the first electrode 21 and the wire 71 to be severed, so that the wire can be melted and is severed.

In this example embodiment, the guide 24 is arranged or designed relative to the end of the first electrode 21 such that a defined spacing a remains between the wire 71 in an end position 25 in the guide 24 and the distal end of the electrode 21. In other words, for direct heating of stent wires in accordance with the foregoing general description of the disclosed embodiments, the distance between the end position 25 of the guide 24 and the distal end of the “active electrode” 21 is zero or even negative, i.e. such that a stent wire 71 located in the end position touches the electrode in an electrically conductive manner or is pressed against the electrode.

For indirect heating of stent wires in accordance with the foregoing general description of the disclosed embodiments, the distance between the end position 25 of the guide 24 and the distal end of the “active electrode” 21 is greater than zero, such that electric arcs L can be produced between a stent wire 71, which is located in the end position 25, and the electrode 21, if a sufficiently high electrical voltage for this purpose is applied between the stent wire and electrode.

In order to form the arcs between the electrode 21 and wire 71, it is necessary for the spacing a between the wire 71 and electrode 21 to be smaller than a spacing b between the first and second electrode 21, 22. The voltage must then be controlled or regulated such that the spacing a between the wire and electrode is sufficient to ignite arcs, while arcs cannot be produced between the electrodes.

The wire 71, which rests on the tubular second electrode 22 in the notch, touches the second electrode directly. For this purpose, it would in principle be sufficient to form the electrode 22 only in the region of the guide 24. However, provision is in this case made for the second electrode 22 substantially to form the effector sleeve or the distal end of the instrument (wherein an insulation layer can be provided toward the outside, as discussed hereinbefore). If only the region of the guide 24 is embodied as the (second) electrode 22 (for example an annular electrode), there is no need to adhere to an exact spacing ratio (spacings a and b) within the effector 20.

A protective gas, for example argon, can be supplied through the lumen 14, so that the arcs ignite in a protective gas atmosphere. This leads to a gentler working sequence and any tissue burn, uncontrolled gas deflagration, etc. may be substantially avoided.

FIG. 3 illustrates a side view of the distal end 11 of the instrument according to another disclosed embodiment of the instrument 10. This embodiment corresponds substantially to that according to FIG. 2. However, in this case, the protective means 23 includes an extended region. This region is provided as a means 27 for threading the wire 71 into the protective means 23 and/or for separating and/or setting apart the wire 71 from the tissue.

This means 27, 28 for threading the stent wires into the guide or very generally into the protective means at the distal end of the sleeve, as shown for example in FIGS. 3 and 4, is beneficial because operators view the effector 20 generally from the proximal end and they accordingly have no direct view onto the distal end of the effectors 20 and because it can be difficult to receive stent wires 71 tightly abutting tissue in the protective means 23 or in the guide 24.

Referring to FIG. 3, means 27 may be configured in a spatula-shaped, finger-shaped or similar manner such that this means 27 can be pushed between stent wires and tissue against which they are abutting, until the particular stent wire has reached the end position 25 in the guide 24. It goes without saying that these spatula-shaped or finger-shaped or similarly shaped means 27 can be adapted in their shape and their size to the various existing and future models of stents. Means 27 as shown in FIG. 3 are handled in particular in the axial direction of the instrument.

Another example embodiment of a means 28 for threading stent wires into the guide is shown in FIG. 4. This means 28 is configured in a helical or corkscrew-shaped manner. In this way, stent wires 71 can be received in the guide 24 and brought into the end position 25 by rotation of the instrument 10. If appropriate, this can be brought about by only rotations of the means 28. The instrument 10 or at least the effector 20 are therefore screwed in under the corresponding wire 71.

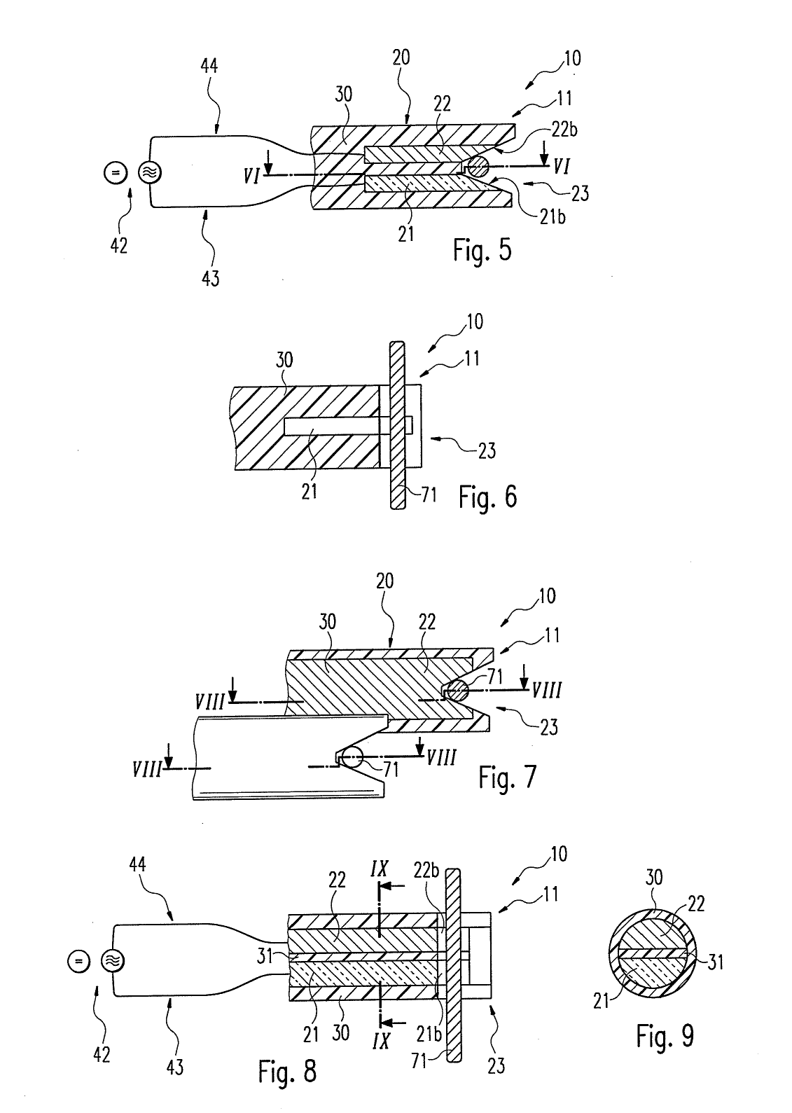

FIG. 5 illustrates a cross sectional view of the distal end 11 of the instrument 10 according to an additional disclosed embodiment of the instrument 10. The effector 20, which is cylindrical and includes the holder, is made of insulating material and includes two mutually opposing electrodes 21, 22 embedded into the insulation layer 30. The protective means 23 is in this case embodied in a manner similar to that according to FIG. 1 or FIG. 2. The electrodes 21, 22 are embedded into the effector 20 such that they each form an active region 21b, 22b in the region of the guide 24. In other words, the electrodes 21, 22 include an active surface for machining the wire that is accessible from the effector 20 or the instrument. The effector 20 therefore forms a holder for the electrodes 21, 22. This embodiment also indicates that the machining of the wire is possible both with alternating current and with direct current. The wire is situated between the electrodes and abuts the active regions of the electrodes and can thus be heated and severed.

A lumen may also be provided between the electrodes 21, 22, so that a protective gas, such as for example argon, could be rinsed against the active regions.

FIG. 6 illustrates a cross-sectional view of the distal end of the instrument 10 along the line VI-VI of FIG. 5. This view illustrates particularly clearly the embedding of one of the electrode 21 into the insulation layer 30.

FIG. 7 illustrates a cross-sectional view of the distal end 11 of yet another disclosed embodiment of the instrument 10. The two halves of the effector 20 are illustrated such that at least one of the electrodes 22 is visible. The electrode 22 is embedded into the effector 20, which is embodied as a holder, such that it is surrounded by insulating material, i.e. the insulation layer 30. The same applies to the opposite electrode (not visible in FIG. 7), the two electrodes being separated from one another by a further insulation layer 31. The further insulation layer cannot be seen in FIG. 7 and would be arranged in the second (front in FIG. 7) half of the effector 20.

The other electrode 21 and insulation layer 31 may be seen in FIG. 8, which illustrates a cross-sectional view of the distal end of the instrument according to FIG. 7 along the line VIII-VIII of FIG. 7). FIG. 9 also illustrates the construction of the electrode arrangement 21, 22 in the effector 20. FIG. 9 illustrates a cross-sectional view of the distal end 11 of the instrument 10 according to FIG. 7 or 8 along the line IX-IX from FIG. 8.

In this embodiment the wire 71 touches the two electrodes 21, 22 as soon as it rests in the notch 24 and thus in the protective means 23. The path of the current therefore extends from one electrode 21 directly into the wire 71, through the wire and from it to the other electrode 22.

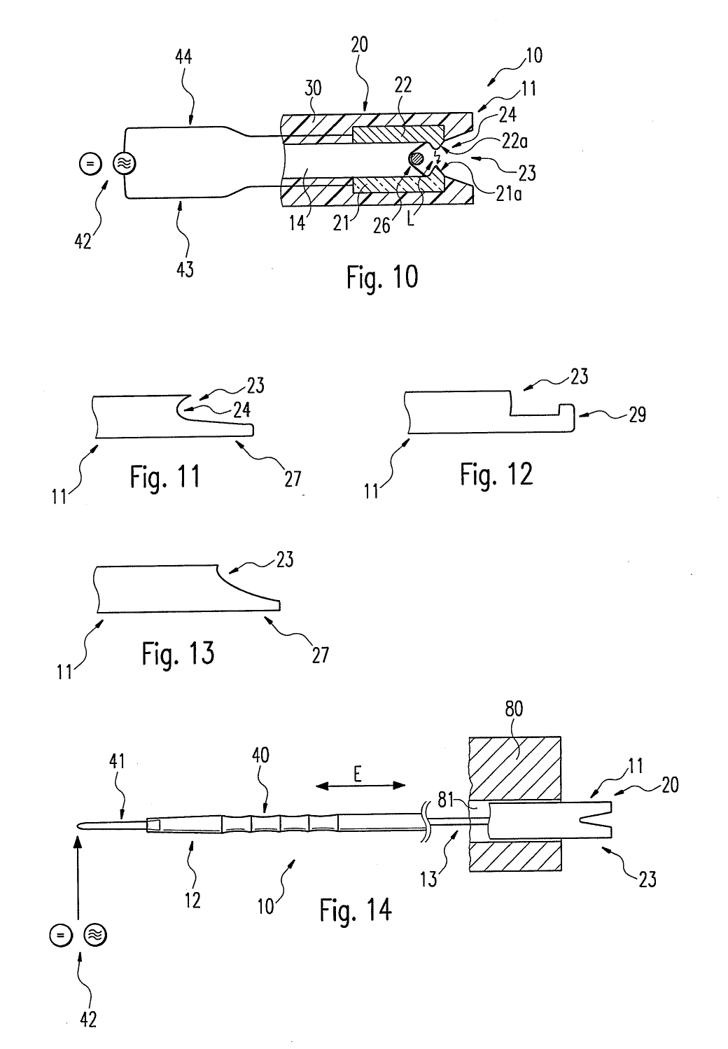

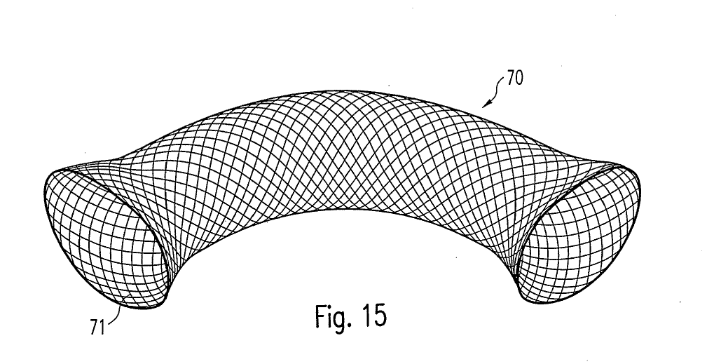

FIG. 10 illustrates a cross-sectional view of the distal end 11 of another disclosed embodiment of the instrument 10. In this case, the effector 20 is embodied as a sleeve or as a pipe with an insulation layer 30 (i.e. the insulation layer forms the sleeve) or the instrument is embodied as a pipe or tube. The two electrodes 21, 22 are embedded into the insulation layer such that they diametrically oppose one another. The pipe forms a lumen 14 which is at least partly surrounded by the electrodes or the active regions 21b, 22b thereof.

The two electrodes 21, 22 each have a raised region (electrode tips) 21a, 22a extending in the direction toward the respectively opposing electrode to form the arcs L. These tips are each arranged at the distal end of the electrodes and form the active regions. This allows arcs to be formed even at a relatively low voltage, wherein the arcs L can be embodied in a controlled manner at a targeted location. In this example embodiment the arc is to be utilised primarily for generating heat, so that a wire 71 is meltable even if it is not directly touched by the electrodes 21, 22 and/or the arc L.

In this embodiment, the guide 24 has a mount or a holding element 26 which is arranged after the electrode tips in the direction of the proximal end 12 of the instrument 10 such that the arc L is not directed directly onto the wire 71. Instead, the wire is merely positioned in proximity to the arc. The arrangement can also be provided as a cut-open or cut-into pipe. The end of the pipe is therefore includes an incision such that the wire can be stored in this incision (which then serves as the mount). Thus, predominantly the heat of the arc is utilised for melting the wire 71. The lumen 14 of the effector 20 can be utilized for supplying protective gas to the electrodes 21, 22, so that the arcs are formed under a protective gas atmosphere. The wire or plurality of wires can be otherwise positioned. It should merely be borne in mind that the heat of the arc can be utilized. If an arc is not to be directly directed onto a metallic wire, then the spacing between the electrodes or the electrode tips and between the electrodes and the wire must be designed accordingly. Otherwise, this embodiment is particularly suitable for non-metallic wires.

It should also be noted that in the embodiment shown in FIG. 10 the spacing between the electrode tips 21a, 22a affords a sufficiently large passage for receiving the wire 71 in the guide 24 or in the mount 26.

The current supply means 43, 44 indicate that both direct current and alternating current can be utilized for machining the stents.

FIGS. 11 to 13 illustrate various means for threading-in, such as have been described hereinbefore in greater detail. The spatula-shaped configurations 27 allow a stent wire to be raised from the tissue in a simple manner. The embodiment according to FIG. 13 allows threading into a guide with a mount or holding element 26, such as is shown in FIG. 10. FIG. 12 illustrates a means or protective means with an explicit holding means 29. The holding means 29 is, for example, a hook element for receiving and securing the wire 71, a plurality of wires, the stent fragment or the stent. In other words, a means 29 is provided, which prevents for example a wire 71, once received or threaded in, from slipping out of the protective means 23 or the means 27, 28 for threading in and/or separating and/or setting apart the wire from the tissue. For this purpose, the protective means 23 can have as the holding means 29 at least one hook element (e.g., a barb) which ensures secure holding of the wire in the protective means 23. The barb therefore allows wires to be “trapped” and drawn away from the tissue. If appropriate, the holding means 29 could be movable relative to the instrument 10. Thus, the wire 71 could be purposefully drawn into the guide 24 and secured therein. The explanting of a wire or fragment is also made possible by the holding means. A second instrument for removing the machined wires from the hollow organ could then be dispensed with.

FIG. 14 illustrates a detail of the instrument 10 according to the disclosed embodiments with the gripping means 40. The instrument 10 is shown being guided in a working channel 81 of an endoscope 80. The gripping means 40 at the proximal end 12 of the instrument 10 has a current connection element or a current connection means 41 via which the two electrodes 21, 22 can be connected to the power source 42. The instrument shown here is, for example, embodied in a similar manner to that shown in FIG. 1.

The instruments according to the disclosed embodiments can be brought up precisely to the stent in the hollow space by using an endoscope.

The instruments according to the disclosed embodiments allow stents in the corresponding hollow organs to be trimmed and thus explanted in a simple manner, and in particular with reduced introduction of current into the tissue surrounding the stent.

It should also be noted that the hatching shown in the figures is not intended to indicate the nature of the material. Thus, for example, one electrode (although generally made of the same material as the other electrode) is illustrated with hatching made up of a broken and solid line, while the other electrode is hatched merely by means of solid lines. This is intended to allow the first and second electrode to be differentiated from each other in the figures. The insulation layers which are necessary for forming the instruments can for example be made of plastic or of ceramic (the hatching of the insulation layers with thick and thin lines generally indicates plastics material, although ceramic can also be provided). In this case, the insulation layers are primarily made of electrically insulating and generally also of thermally insulating material.

It should be pointed out here that all the above described parts and in particular the details illustrated in the drawings are essential for the disclosed embodiments alone and in combination. Adaptations thereof are the common practice of persons skilled in the art.

1-31. (canceled)

32. A bipolar instrument for endoscopically controlled shortening and/or fragmentation of a stent located in the gastrointestinal tract, tracheobronchial system or in other hollow organs, the instrument comprising:
a first electrode and a second electrode arranged at a distal end of the instrument and connected to and receiving current provided by a power source, wherein the first and second electrodes are configured such that at least one wire of the stent can be severed at a particular location; and
protective means connected to the electrode means, wherein the protective means is configured to separate the wire from tissue of the gastrointestinal tract, tracheobronchial system or other hollow organs and/or to secure the wire to the instrument during the severing of the wire.

33. The bipolar instrument according to claim 32, wherein the first and second electrode are configured to pass a current from the power source through the at least one wire of the stent, thereby severing the wire by heating.

34. The bipolar instrument according to claim 32, wherein the first and second electrode are configured to form electric arcs between the first electrode and the at least one wire and/or between the first electrode and the second electrode, thereby severing the wire by heating.

35. The bipolar instrument according to claim 32, wherein the power source provided is a high-frequency AC power source.

36. The bipolar instrument according to claim 32, wherein the power source provided is a DC power source or low-frequency AC power source.

37. The bipolar instrument according to claim 32, wherein the power source is connected to a controller that controls the current provided by the power source for automatically controlled severing of the wire.

38. The bipolar instrument according to claim 37, wherein the controller is connected to an arc monitor and/or a current monitor, wherein the current is controlled as a function of the detected arc or as a function of a detected current value.

39. The bipolar instrument according to claim 37, wherein the controller measures the direct current and is configured to interrupt the supply of current if a limit value is exceeded.

40. The bipolar instrument according to claim 32, wherein at least the first and second electrodes and the protective means are embodied as an effector, wherein the effector is arranged at the distal end of the instrument.

41. The bipolar instrument according to claim 32, further comprising a rigid or flexible shaft or catheter that is configured to be inserted through an instrument channel of a rigid or flexible endoscope.

42. The bipolar instrument according to claim 41, wherein the shaft or the catheter is a pipe or a tube with a respective lumen for supplying a fluid to the electrodes and/or the protective means and/or the hollow organ.

43. The bipolar instrument according to claim 42, wherein the lumen surrounds the first and second electrodes, and the first and second electrodes and/or the protective means can be cooled by the fluid supplied.

44. The bipolar instrument according to claim 42, wherein the lumen surrounds the first and second electrodes, and the fluid supplies a protective gas atmosphere in which the passing of the current through the wire and/or the forming of arcs is carried out.

45. The bipolar instrument according claim 41, wherein the shaft or the catheter is a rod element made of solid material.

46. The bipolar instrument according to claim 41, wherein the shaft or the catheter is made of ceramic, plastic or other insulating material.

47. The bipolar instrument according to claim 32, wherein the protective means is electrically insulating.

48. The bipolar instrument according to claim 32, wherein the protective means is configured such that the wire is held at a predetermined spacing from the first electrode and/or from the second electrode.

49. The bipolar instrument according to claim 32, wherein the protective means further comprises means for threading the wire at least into the protective means and/or for separating and/or setting apart the wire from the tissue.

50. The bipolar instrument according claim 49, wherein the means for threading the wire at least into the protective means and/or for separating and/or setting apart the wire from the tissue allows a plurality of wires to simultaneously be threaded in and/or separated and/or set apart from the tissue.

51. The bipolar instrument according to claim 49, wherein the means for threading the wire at least into the protective means and/or for separating and/or setting apart the wire from the tissue is spoon, finger or spatula-shaped that is pushed or pulled under the at least one wire in a substantially rectilinear movement in the axial direction of the instrument.

52. The bipolar instrument according to claim 49, wherein the means for threading the wire at least into the protective means and/or for separating and/or setting apart the wire from the tissue is screwdriver or corkscrew-shaped that is screwed and/or slid under the at least one wire in a substantially turning or rotating movement.

53. The bipolar instrument according to claim 49, wherein the protective means further comprises at least one guide into which the wire slips and is fixed therein during the pressing-on of the instrument and/or the sliding or turning of the means for threading the wire at least into the protective means and/or for separating and/or setting apart the wire from the tissue and/or of the instrument.

54. The bipolar instrument according to claim 53, wherein the guide is a notch, and wherein the wire is received in the notch.

55. The bipolar instrument according to claim 53, wherein the guide is configured such that the received wire is held at a predetermined spacing from the first electrode.

56. The bipolar instrument according to claim 32, wherein, when the wire is received within the protective means, the spacing between the first electrode and the wire is smaller than a spacing between the first electrode and the second electrode, so that arcs can be formed between the first electrode and the wire.

57. The bipolar instrument according to claim 40, wherein the effector comprises a sleeve for holding the electrodes, wherein the sleeve forms a lumen for supplying a fluid to the electrodes and/or the protective means and/or the hollow organ, and wherein the sleeve is made of insulating material.

58. The bipolar instrument according to claim 57, wherein the protective means is connected securely to the sleeve for holding the electrodes.

59. The bipolar instrument according to claim 42, wherein the first electrode is arranged in the lumen and the second electrode is arranged coaxially with the first electrode and set apart therefrom.

60. The bipolar instrument according to claim 45, wherein the first electrode and the second electrode are embedded, set apart from one another, in the rod element such that they each form an active region at a distal end of the instrument.

61. The bipolar instrument according to claim 42, wherein the first electrode and the second electrode are embedded, set apart from one another, in the pipe or tube such that they each form an active region at a distal end of the instrument and that the active regions at least partly surround the lumen.

62. The bipolar instrument according to claim 34, wherein the first electrode and/or the second electrode each comprise at least one raised region extending in the direction toward the respectively opposing electrode in order to form the arcs.

63. The bipolar instrument according to claim 32, wherein the first and second electrodes are made of a high temperature-resistant material.

64. The bipolar instrument according to claim 63, wherein the first and second electrodes are made of lanthanated tungsten.

65. The bipolar instrument according to claim 49, wherein the protective means and/or the means for threading the wire at least into the protective means and/or for separating and/or setting apart the wire from the tissue comprise a hook for receiving and securing the wire on the instrument.

66. A method for endoscopically controlled shortening and/or fragmentation of a stent located in the gastrointestinal tract, tracheobronchial system or in other hollow organs, with a bipolar instrument comprising a first electrode and a second electrode arranged at a distal end of the instrument, and protective means mechanically connected to the first and second electrodes, the method comprises the steps of:
a) bringing the instrument into the hollow organ and to the stent;
b) separating at least one wire of the stent from tissue of the hollow organ by inserting or screwing in the protective means between the wire and the tissue and/or securing the wire to the instrument by means of the protective means and positioning the at least one wire at least in proximity to the first and second electrodes by means of the protective means such that a current can be passed through the wire of the stent and/or electric arcs can be formed between the first electrode and the at least one wire and/or between the first electrode and the second electrode;
c) passing the current from a power source via the first and second electrodes into the at least one wire and/or forming electric arcs between the first electrode and the wire and/or between the first electrode and the second electrode and thereby severing the wire; and
d) repeating steps b) and c) for shortening and/or fragmenting the stent.
